(19) United States
(12) Patent Application Publication
(10) Pub. No.: US 2022/0370077 A1
(43) Pub. Date: Nov. 24, 2022

(54) JAILED AIRWAY DETECTION AND AIRWAY STENT HOLE CUTTING GUIDE

(71) Applicants: The Cleveland Clinic Foundation, Cleveland, OH (US); New COS Inc., d/b/a VisionAir Solutions, Cleveland, OH (US)

(72) Inventors: Thomas Gildea, Cleveland Heights, OH (US); Keith Grafmeyer, Cleveland, OH (US); Jake Eva, Cleveland Heights, OH (US); Kevin Libertowski, Cleveland, OH (US); Brian Beckrest, Lakewood, OH (US); Tyler Blackiston, University Heights, OH (US)

(21) Appl. No.: 17/747,075

(22) Filed: May 18, 2022

Related U.S. Application Data
(60) Provisional application No. 63/190,356, filed on May 19, 2021.

(51) Int. Cl. A61B 17/12; G06T 7/00

(52) U.S. Cl. CPC A61B 17/12104; A61B 17/12036; A61B 17/12145; A61B 17/1215; G06T 7/0012; G06T 2207/30061

(57) ABSTRACT

A bronchial stent includes a first branch configured to widen, open, and/or mechanically support a first airway; an obstructive portion that, when the stent is deployed in the first airway, obstructs a second airway, the second airway forming a branching connection with the first airway; and a feature proximal to the obstructive portion, the feature configured to facilitate opening of the obstructive portion.

### CROSS-REFERENCE TO RELATED APPLICATION(S)

This application claims priority to and any benefit of U.S. Provisional Application No. 63/190,356, filed May 19, 2021, the content of which is incorporated herein by reference in its entirety

### FIELD

This disclosure relates to airway stents and stent design. It relates more generally to surgical planning and to devices, systems, and methods for model-based stent design and placement.

### BACKGROUND

Tracheobronchial protheses, also known as airway stents, support airway structural integrity where there has been tracheal collapse. They are typically deployed bronchioscopically and have tube-like shapes that mirror the interior of the airway as closely as possible for maximum effectiveness.

Traditionally manufactured stents have often fit poorly. They tend to be mass produced with an averaged shape to fit an average airway structure, yet actual airway shapes vary substantially. Poorly fitting stents can cause airway occlusion and scar tissue formation. They can dislodge and move within the airway, potentially cutting off other portions of it. Sometimes, they can even cause infection.

It is now possible to design these stents in silico, or by computer, to fit a particular airway in a particular patient. Computed tomography (CT) scans and/or magnetic resonance imaging (MRI) scans provide accurate three-dimensional (3D) representations of the patient's airway. Software uses the data from the scans to design stents that can better represent the shape of the imaged airway. 3D printing techniques generate a stent with that precise shape.

Nonetheless, these techniques still have notable limitations. In particular, fabricating and deploying computer-designed, 3D printed stents without the unintended consequence of “jailing off” or blocking an air passage is often impossible. Well-placed holes in the stent can address this drawback by restoring airflow to blocked passages. However, there are currently no accurate and reliable ways to do this. Physicians must guess at hole locations based on static measurements during the implantation or deployment procedure.

### SUMMARY

Aspects of the present disclosure include a bronchial stent comprising a first branch configured to at least one of widen, open, and mechanically support a first airway, an obstructive portion that, when the stent may be deployed in the first airway, obstructs a second airway, the second airway forming a branching connection with the first airway, and a feature proximal to the obstructive portion, the feature configured to facilitate opening of at least a part of the obstructive portion.

The feature may form one of a circumference of and an outline of the at least a part of the obstructive portion. The stent may have an average thickness and the feature may have a thickness greater than the average overall stent thickness. The feature thickness may be at least one of ten percent more than the average overall stent thickness, twenty percent more than the average overall stent thickness, fifty percent more than the average overall stent thickness, twice the average overall stent thickness, three times the average overall stent thickness, and four times the average overall stent thickness. The feature may comprise a raised portion of the stent. The feature may comprise a perforation. The perforation may substantially outline the at least a part of the obstructive portion. The facilitating opening of at least a part of the obstructive portion may comprise facilitating mechanical removal of the feature from the stent.

The mechanical removal may comprise punching the obstructive portion with an instrument. The instrument may be a forceps. The removal may be performed prior to deploying the stent in the first airway. The stent may be made from a material and the feature may comprise the stent material. The stent may be made from a material and the feature may comprise a hole in the stent. The hole may substantially overlap the obstructive portion. The hole may be the at a least part of the obstructive portion. The hole may substantially encompass the obstructive portion. The material may be silicone. The stent may be 3D printed. The first and second airway may belong to a patient, and the first branch, the obstructive portion, and the feature proximal to the obstructive portion are configured to substantially fit at least the first airway. The configuring of the first branch, the obstructive portion, and the feature proximal to the obstructive portion may comprise designing the first branch, the obstructive portion, and the feature proximal to the obstructive portion using computer aided design (CAD). The CAD may use at least one of CT image data and MRI image data of the first and second airways. The feature may be proximal to an edge of the stent.

Configuring the feature to facilitate opening of at least a part of the obstructive portion may comprise designing the feature to avoid creating bridge-like portions in the stent. Designing the feature to avoid creating bridge-like portions may comprise creating a notch at the edge of the stent. The first branch, the obstructive portion, and the feature proximal to the obstructive portion may not be designed to fit a class of patients. The stent may comprise a reinforcing feature. The reinforcing feature may have a thickness greater than an average overall stent thickness. The reinforcing feature may be configured to obstruct at least one of the second airway and a third airway.

The stent may comprise a fitting portion, the fitting portion configured to accommodate another stent. The accommodating another stent may comprise at least one of fitting into a hole in the other stent, connecting to a connecting portion of the other stent, and encompassing an encompassing portion of the other stent. The accommodating another stent may comprise creating an air-tight seal between the stent and the other stent. The accommodating another stent may be accomplished while the stent is inside a patient. The accommodating another stent may create a stent architecture comprising the stent and the other stent. The stent architecture may comprise more than two stents. The stent may comprise an opening configured for a therapeutical purpose. The therapeutic purpose may comprise delivery of medicine.

Aspects of the present disclosure may comprise a method of creating the stent comprising designing the first branch, the obstructive portion, and the feature proximal to the obstructive portion using CAD, and 3D printing the stent. The designing may use CAD tools. It may use other software tools in conjunction with CAD. It may comprise using at least one of CT image data and MRI image data of the first and second airways. The CAD may design the stent to fit portions of a specific patient. The CAD may be used to segment at least one image of a region of interest in a patient to provide a three-dimensional model representing at least a portion of the first airway and at least a portion of the second airway, then select from a plurality of locations within the airway model and a corresponding plurality of diameters for the plurality of location, and finally construct a stent model from the selected locations and diameters.

The method may comprise generating the stent model as a cylindrical mesh that extends from a first location of the plurality of locations to a second location of the plurality of locations following a centerline of the three-dimensional airway model, with a diameter of the cylindrical mesh at a given point between the first location and the second location being a function of a first diameter associated with the first location, a second diameter associated with the second location, a distance between the first location and the second location, and a distance of the given point from the first location. The method may comprise placing a diagnosis marker, representing a stricture in at least one of the first airway and the second airway, the model generator selecting a thickness for at least a portion of the stent model according to a location and identity of the diagnosis marker. The segmenting may comprise segmenting the at least one image via a machine learning (e.g., convolutional neural network trained on images segmented by a human expert), the convolutional neural network receiving the at least one image of the region of interest and providing the three-dimensional airway model as an output. The method may comprise editing the stent model via graphical user interface to change one of a thickness of the stent model and a diameter of the stent model at the selected point.

Aspects of the present disclosure may further comprise a system comprising a processor, and a non-transitory memory storing computer executable instructions for performing the methods disclosed herein.

### BRIEF DESCRIPTION OF DRAWINGS

FIG. 1 shows a system 100 that may be used in computer aided design (CAD) for generating models of stents and for 3D printing those stents according to aspects of the present disclosure.

FIG. 2A shows how model generator 116 in system 100 generates an initial stent model 200.

FIG. 2B shows an exemplary 3D printed real-world stent 260 according to aspects of the present disclosure.

FIG. 2C shows obstructive portion 206 from the perspective of jailed airway 260 according to aspects of the present disclosure.

FIG. 2D is a close-up view of obstructive portion 206 and jailed airway 260.

FIG. 3A shows a first option 300 to address a jailed airway within the scope of the present disclosure.

FIG. 3B shows how hole 310 may be sized and positioned to be precisely coincident with obstructive portion 206.

FIG. 3C shows another variation in which hole 312 is centrally placed within obstructive portion 206 but is smaller than obstructive portion 206.

FIG. 3D shows a large hole 314 creating a bridge-like structure according to aspects of the present disclosure.

FIG. 3E shows one way to address problems associated with bridge-like structure 202 according to aspects of the present disclosure.

FIG. 3F shows a notch solution to the problem of bridge-like structure 202f according to aspects of the present disclosure.

FIG. 4A shows another feature 400 that can assist in opening the stent 200 to flow from jailed airway 260 according to aspects of the present disclosure.

FIB. 4B shows feature 400 in more detail.

FIG. 4C shows how ring 400a can be substantially coincident and circumferential with respect to obstructive portion 206.

FIG. 4D shows how ring 400a can be larger than obstructive portion 206.

FIG. 4E shows how ring 400 would be placed proximally to edge 202e such that at least part of portion 400c overlaps with edge 202e.

FIG. 4F shows another version 410 of a ring construction with perforation according to aspects of the present disclosure.

FIG. 5A shows one way a thickness of the printed walls of the stent 200 can vary according to aspects of the present disclosure.

FIG. 5B shows an alternative method of increasing the thickness of stent according to aspects of the present disclosure.

FIG. 6 shows a method of generating a stent for a patient's airway according to aspects of the present disclosure.

FIG. 7 presents a method 700 of printing, preparing, and deploying a stent according to aspects of the present disclosure.

### DETAILED DESCRIPTION

The present disclosure merely exemplifies the general inventive concepts using specific variations. Variations encompassing the general concepts may take various forms. The general concepts are not intended to be limited to the specific variations described herein.

As used herein, the term “model” can refer to a representation of an object created on a computer. In some instances, the model can be a three-dimensional representation of the object.

The term “coordinate system” can refer to a system of representing points in a space of given dimensions by coordinates.

As used herein, the terms “subject” and “patient” can refer, interchangeably, to any warm-blooded organism including, but not limited to, a human being, a pig, a rat, a mouse, a dog, a cat, a goat, a sheep, a horse, a monkey, an ape, a rabbit, a cow, etc.

### Overview of Exemplary System for Generating Modeled Stents

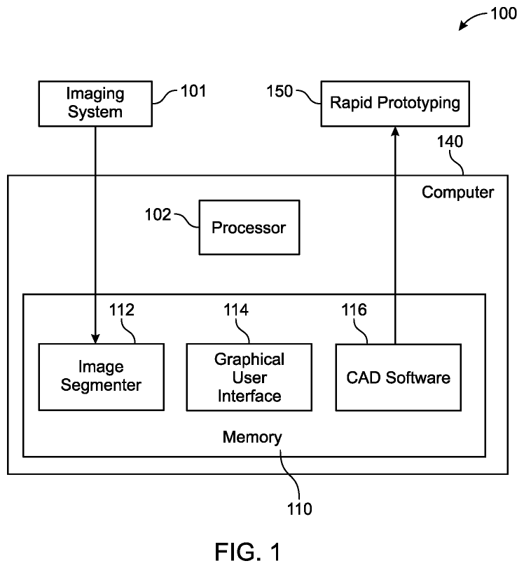

FIG. 1 shows a system 100 that may be used in computer aided design (CAD) for generating models of patient-specific (and other) stents and for 3D printing those stents, according to aspects of the present disclosure. As shown in FIG. 1, system 100 may be implemented by one or more computers 140, which may be general purpose computers, computers adapted specifically for the purposes of CAD, and/or other programmable data processing apparatus. Suitable computers may include graphics modules and/or artificial intelligence (AI) modules particularly suited for graphical design. Computer 140 may include various input devices (not shown) such as one or more of a touchscreen, a mouse, a trackball, a keyboard, a microphone, and a gesture recognition interface. Computer 140 can include an output device (not shown), for example, one or more of a display, a speaker, and a printer.

System 100 includes at least one processor 102 and at least one non-transitory memory 110 storing CAD software 116 as well as executable instructions for designing airway stents. Non-transitory medium 110 can include any medium that is not a transitory signal and can contain or store the program for use by or in connection with the instruction or execution of a system, apparatus, or device. For example, non-transitory medium 110 can be an electronic, magnetic, optical, electromagnetic, infrared, semiconductor system, apparatus or device, a portable computer diskette, a random access memory, a read-only memory; an erasable programmable read-only memory (or Flash memory), or a portable compact disc read-only memory.

CAD software 116 may include any software suitable for providing components of a stent design tool described below that are executable by processor 102, including an image interface for interfacing with imaging system 101 and image segmenter 112, and graphic user interface (GUI) 114. It may further include the image segmenter 112 and a model generator.

The executable instructions include image segmenter 112 that can segment images from imaging system 101 to provide three-dimensional models of the objects in those images. In one implementation, the imaging system 101 is a CT imaging system that provides one or more CT images to the imager interface, although it will be appreciated that the imaging system 630 can comprise any imaging system capable of providing three-dimensional models of an airway of a patient. In another variation, imaging system 101 is an MRI imaging system. Imaging system 101 may include multiple types of images and imaging systems (e.g., both CT and MRI). The images provided to the segmenter 112 may be a series of two-dimensional images from which a three-dimensional model of the airway of the patient can be constructed. They may also include some three-dimensional images.

Segmenter 112 may take an image of an airway, segment that image, and generate a 3D model of at least a portion of the airway. Often, the stent model will be based on the segmented image model of a particular patient's airway. However, this need not always be case. In some cases, the stent model may be more general and not specifically designed for any one patient. System 100 can display the airway model to the user via GUI 114. Image segmenter 112 may also generate a 3D model of a stent design for display on GUI 114. The image segmenter 112 can use any appropriate means for determining the boundaries of the airway within the received images. In one implementation, the image segmenter 112 includes a convolutional neural network, trained on CT images that have been segmented by a human expert, that produces the segmented airway model from the received CT images. The user may use the GUI 114 to edit and manipulate the design in real time.

The image segmenter 112 can utilize any suitable algorithm for segmenting input images. These algorithms include, for example, machine learning models. The machine learning models may be trained on example images segmented by human experts. Once trained, the models can provide the three-dimensional airway model based on input two- or three-dimensional images.

Suitable machine learning systems for this purpose include convolutional neural networks, recurrent neural networks, other neural networks, decision trees, and generalized adversarial networks. Use of other approaches such as energy minimization, clustering, and edge detection algorithms, may also generate the 3D models.

These aforementioned models and systems may be configured to automatically generate a stent model based on the 3D models of the airway. In addition, system 100 also includes human/user input in stent design. User input is largely facilitated by GUI 114. The GUI 114 may provide controls to allow the user to rotate or zoom in or out from the model. This may facilitate design of the stent and/or visualization of the airway. In particular, GUI 114 may allow a user, such as a physician or technician, to determine a placement and size of the stent within the airway. Models may provide reference points or points of interpretation to aid user design. For example, the models may calculate certain reference locations within the stent for the user (e.g., geometric center, centerline, maximum thickness, minimum thickness, branching points of the stent, etc.). These reference locations may be based on the construction of the airway to facilitate an accommodating design. Examples of the latter include, for example, a point in the stent at which multiple branches of the airway meet.

Typical user stent design would proceed by a user, for example, first using the GUI 114 to select locations within an airway model generated from images by the segmenter 112. Once selected, the user could assign a number of parameters (e.g., diameters, thicknesses, or features such as holes and perforations) for the stent model at each of the plurality of locations. Data can be entered manually and/or graphically. The GUI 114 may prompt the user to, for example, select four initial locations and corresponding diameters, representing a proximal end of the stent, a primary distal end of the stent, a secondary distal end of the stent, and a join location for first and second branches represented by the primary and secondary distal ends of the stent. This initial selection is merely exemplary. Initial selection can include more or fewer initial locations.

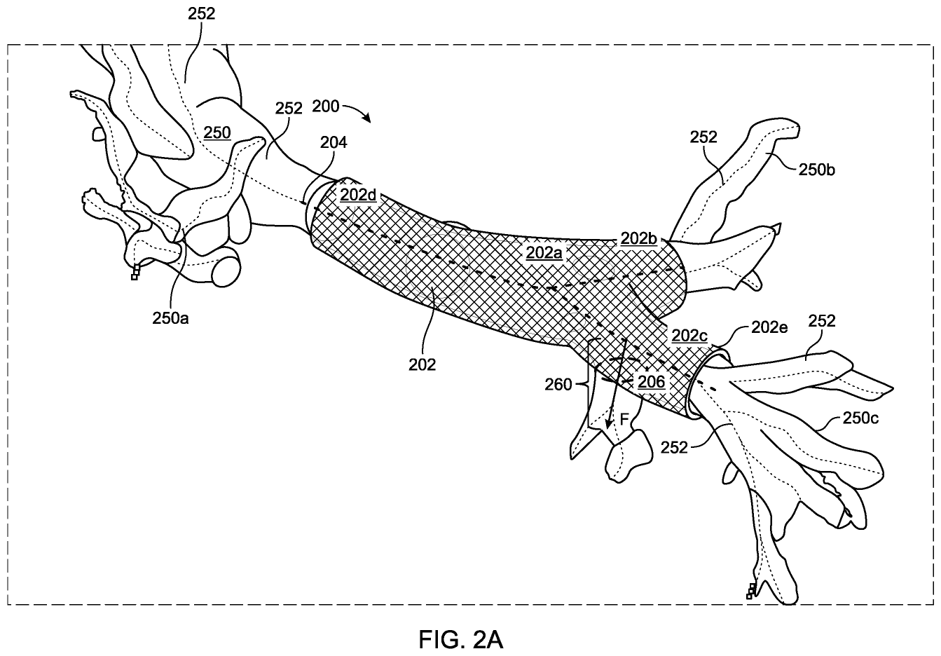

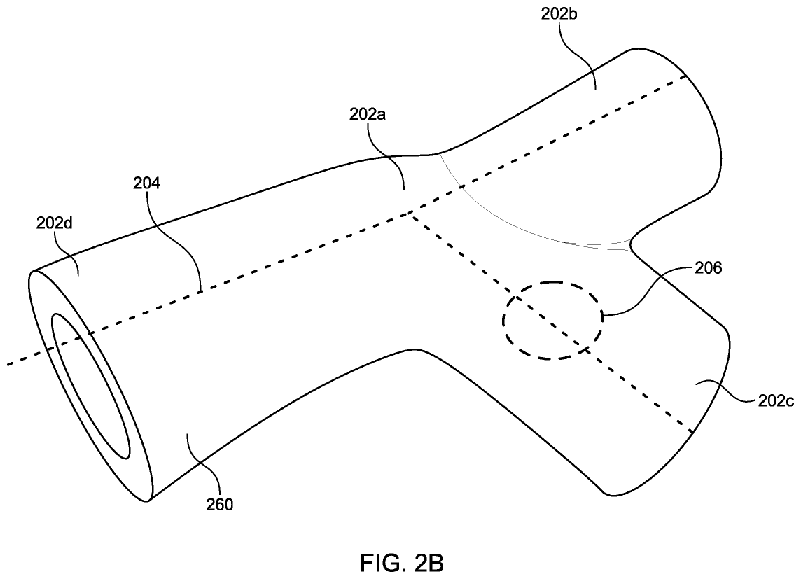

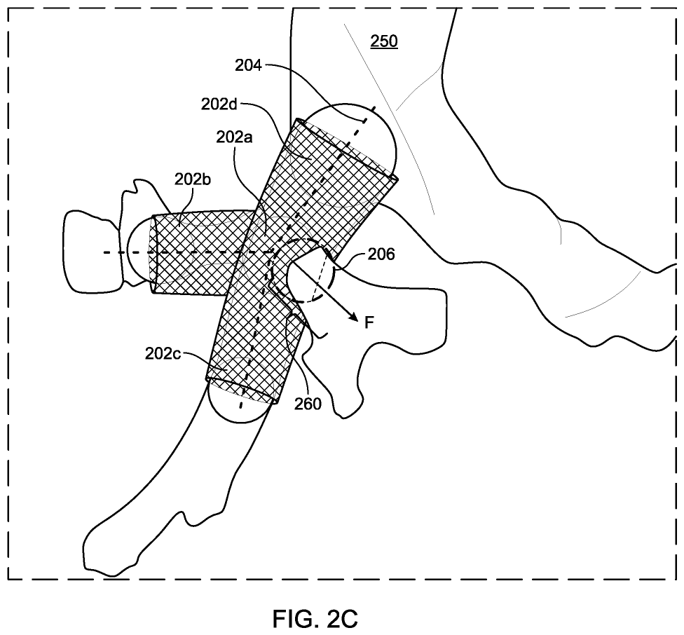

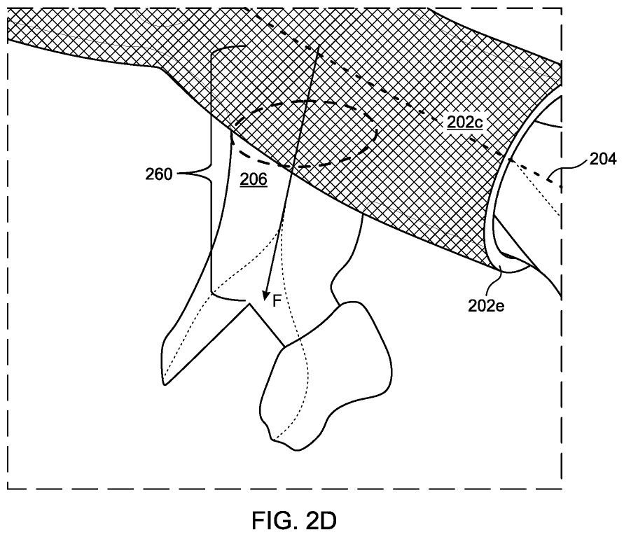

Once the initial locations are selected, CAD software (e.g., a model generator) 116 may construct a virtual stent model based on the selections and inputs. In one example shown in FIG. 2A, the model generator 116 generates an initial stent model 200 by representing user selected locations and associated input diameters as the base of a cylinder and connects the locations using a cylindrical mesh 202. The model 200 may be fit using only user specified locations in the 3D airway image 250. Alternatively, it may be fit according to a combination of user input and data from the 3D airway image 250. A diameter of the stent may be a weighted linear combination of the selected first and second diameters, with the weights for each point determined from the distances from that point to each of the first and second ends of the stent. For example, the CAD software 116 can determine the diameter, dₚ, at a given point between the first location and the second location as dₚ=d₁+(d₂−d₁)(l_{1,p}/l_{1,2}), where d₁ is the first diameter, d₂ is the second diameter, l_{1,2} is the distance between the first location and the second location, and l_{1,p} is the distance between the given point and the first location.

In one implementation, an approximate centerline (e.g., line 250 in FIG. 2A discussed below) of the stent model is created. The centerline may track a centerline of the model of the patient's airway. For a branching stent, at each end location, a cylindrical mesh with the selected diameter can be generated with the cylindrical meshes meeting at the fourth location. In one implementation, the cylindrical mesh follows the centerline of the airway, although other algorithms, such as a spline approach, can be applied to a set of points selected on the three-dimensional airway model to generate the centerline and diameter of the cylindrical mesh at each point based on the selected locations and diameters. Models may apply smoothing to avoid rapid deviation in the centerline of the stent present in the image model. A diameter of the cylindrical mesh 202 at points between the selected locations (not shown) can be determined, for example, via a polynomial or spline interpolation between the two locations.

The initial stent model may be displayed to the user via the GUI 114 for editing. In one example, the user selects additional locations (e.g., locations 202a, 202b, 202c, and 202d) within the initial stent design 202. The user then changes stent parameters (e.g., diameter, thickness, presence of a hole, etc.) at each location 202a, 202b, 202c, and 202d. For example, the user may add additional branches to the stent (e.g., a new branch at location 202b) if an airway branch is not mirrored in the original stent model 200. The user may also change angles of branches at selected locations within the initial stent design 300. Branches can be color coded to alert the user to the branch selected for editing. The stent thickness, inner diameter, and outer diameter can also be viewed at a selected point and edited via GUI 114, either by directly entering a value, in which case the inner stent diameter remains fixed and the outer diameter is adjusted, or by changing either or both of the inner and outer stent diameters at a given point. The thickness of the stent model can also be adjusted globally.

In one implementation, users can place markers in the airway representing conditions within the airway that could cause stricture within the airway. In response to these markers, a thickness of the stent could be altered, based on the specific diagnosis at each region. For example, a tumor growing in the airway will require more radial force to hold it open that a disease that causes inflammation in the airway tissue. Each diagnosis can have a default stent wall thickness and width, representing a length of the stent that should be altered in response to a given diagnosis marker, that is used by the CAD Software 116 to generate the initial model, and the user can alter the thickness in the initial model via the GUI 114. Once the user has finished editing the stent model, the user can approve the model via the GUI 114.

The approved model can be provided to a manufacturer via a network interface or provided to a rapid prototyping system 150, such as a 3D printer, to obtain a stent for use in the patient's airway. Any suitable 3D printer may be used. For example, suitable 3D printers include those that can print 3D objects using polymers such as silicone. Examples include those using a material jetting process. Additive manufacturing techniques can also be employed in stent fabrication. In addition, stents may be made from models in the context of the present disclosure in ways other than by 3D printing the stents themselves. For example, users may 3D print the negative of the stent model to create 3D molds. This may allow more flexibility in stent materials since moldable materials are not necessarily 3D printable. Moreover, 3D printing materials beyond those best for stents may be used to make the mold (e.g., ceramics and or carbon-based materials, other polymers).

An exemplary 3D printed real-world stent 260 is shown in FIG. 2B. Real-world stent 260 has approximately the same shape as stent model 200 in FIG. 2A. In particular, FIG. 2B shows centerline 204 and positions 202a-202d of stent model 200 superimposed on the photograph of stent 260 for comparison. Exemplary real-world stent 260 was 3D printed using silicone material. Silicone is a polysiloxane polymer that can be advantageous in stent applications because of the ease and accuracy with which it is 3D printed, its high biocompatibility, flexibility, relatively inert chemistry, and the fact that it does not support microbiological growth. Another property that may be advantageous, particularly during endoscopic deployment, is its transparency. Although stent 260 is made of 3D printed silicone, it is to be understood that this is merely exemplary. Any suitable, printable, and/or moldable stenting material may be used. Such materials include other polymers with high biocompatibility, flexibility, high tear strength, toughness, elongation, elasticity, and printability.

### Design Considerations Regarding Jailed Airways

1. Jailed Airways Created in Modeling Patient-Specific Stents

Turning back to FIG. 2A, stent model 200 may create a jailed airway 260. Jailed airway 260 is a portion of airway 250 that has been walled off or sequestered from the rest of airway 250 by the stent 200, specifically by the stent 200's obstructive portion 206. As shown in FIG. 2A, jailing off airway 260 would prevent flow F from airway 250. This may essentially forfeit use of jailed airway 260 in normal respiration. FIG. 2C shows obstructive portion 206 from the perspective of jailed airway 260. In FIG. 2C, obstructed flow F is out of the page. FIG. 2B shows an approximate location of obstructive portion 206 in real world stent 260.

FIG. 2D shows a closeup of jailed airway 260 and obstructive portion 206 of stent 200. If obstructive portion 206 remains when the stent 200 is deployed in the actual patient airway represented by 250, it would prevent flow F of air to and from jailed airway 260 and the rest of the airway 250. There may be some instances in which jailing an airway is advantageous (e.g., if the airway 260 is non-functional and/or exhibits a pathology that interferes with the rest of airway 250). In many instances, however, it is advantageous to utilize as many airways as possible in respiration. Therefore, in many instances, it would be advantageous to redesign stent 200 so that airway 260 is no longer jailed.

2. Precision Design and Placement of Stent Holes to Allow Flow into Jailed Airways

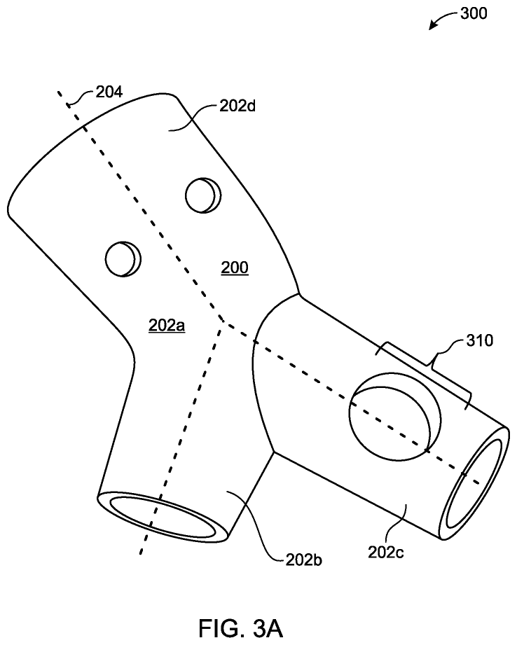

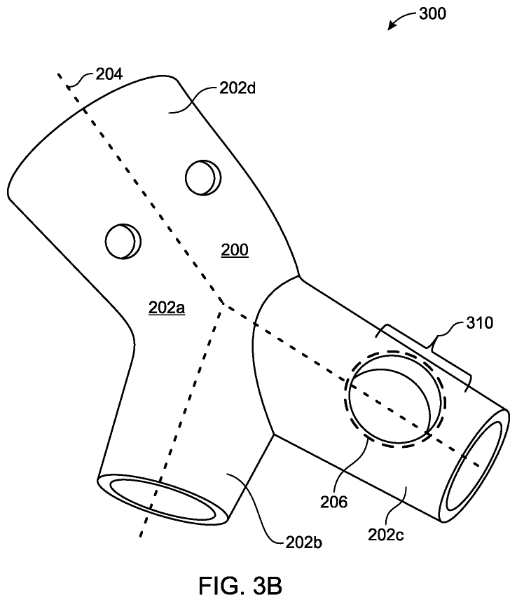

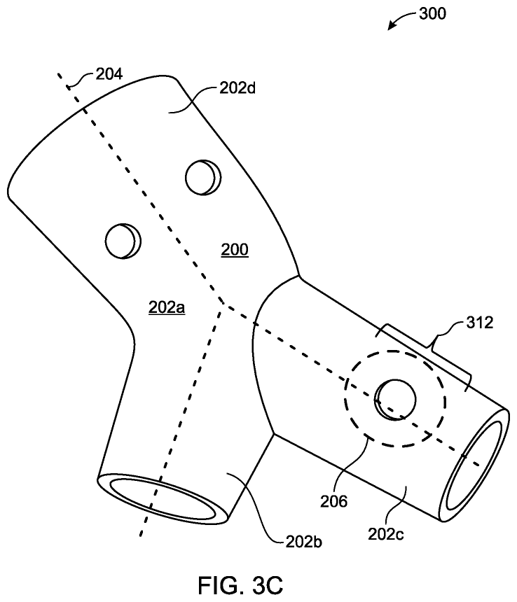

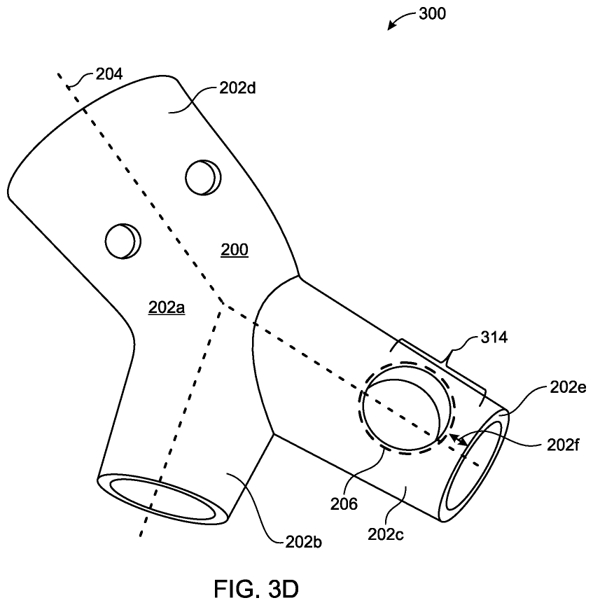

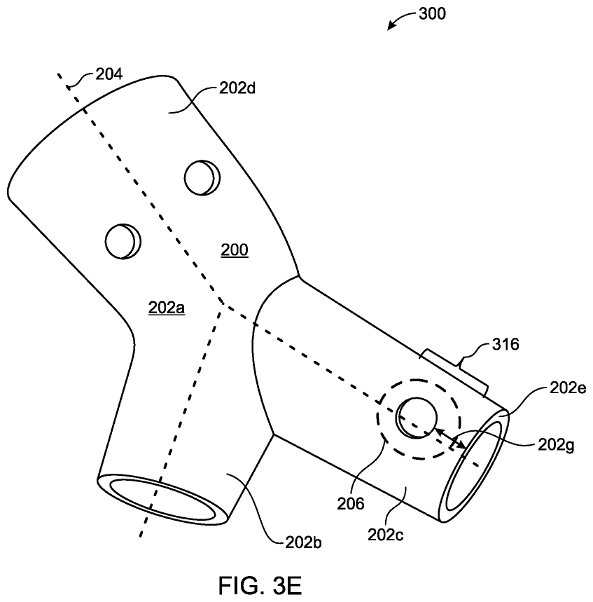

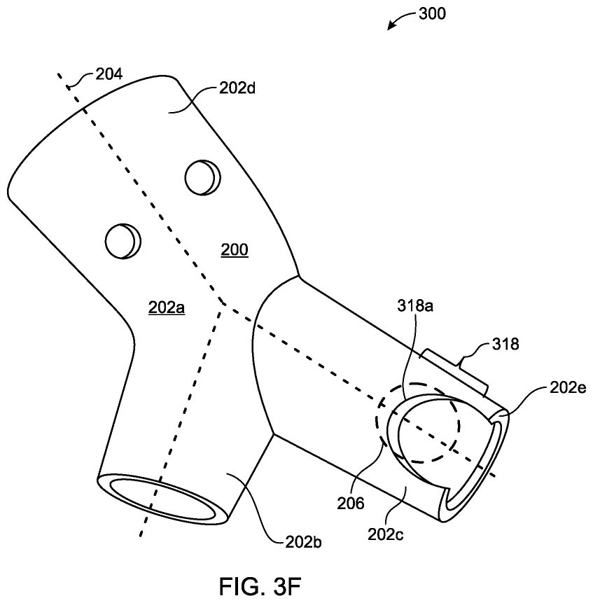

A number of options are available to the user to address jailed airway 260. FIG. 3A shows a first option 300. In option 300, stent 200 is redesigned to have a hole 310 in place of obstructive portion 206. Hole 310 can be designed into the stent model 200 before the stent is printed to form a real-world stent (e.g., stent 260). In this way, stent 200 can simply be 3D printed (e.g., in silicone, as described in the context of FIG. 2B above) having hole 310 in the precise position that the model airway 250 indicates for the interface between stent 200 and jailed airway 260 (i.e., obstructive portion 206 of stent 200). As shown in FIG. 3B, hole 310 may be sized and positioned to be precisely coincident with obstructive portion 206. Precision sizing can be facilitated by the modeling process prior to 3D printing. The size of the hole depends entirely on the application. Generally, it may be advantageous to have larger holes, e.g., to support greater flow F between airway 250 and formerly jailed airway 260. Therefore, having the hole as large as the obstructive portion of the stent (e.g., hole 310 which is the same size as obstructive portion 206 (FIG. 3B)) can be advantageous. In some cases, it may be advantageous for the hole to be even larger than obstructive portion 206, again most likely to facilitate maximum air flow.

FIG. 3C shows another variation in which hole 312 is centrally placed within obstructive portion 206, but is smaller than obstructive portion 206. This example is meant to show that there is no specific requirement for hole sizing within the model 200. In some cases, restricted airflow may be advantageous so as, for example, not to have edges of the hole rub and grind into the jailed airway branch point (e.g., carina). In those cases, smaller holes such as 312 (FIG. 3C) would be preferable.

FIG. 3D shows another instance where a smaller hole may be preferable. FIG. 3D shows a hole 314 sized to fit the obstructive portion 206. However, the location of the obstructive portion 206 in FIG. 3D is relatively close to the edge 202e of the stent 200. Note that in FIG. 3D the location of obstructive portion 206 has been moved closer to edge 202e than its position in FIG. 2A in order to facilitate discussion. In this case, a bridge-like structure 202f of stent 200 is formed that is relatively thin and fragile. Such bridge-like portions 202f, especially if made from silicone, may easily break and/or tear when stent 200 is deployed in a patient's airway. Fragments of a broken bridge-like structure 202f may be dangerous, uncomfortable, or irritating to the patient.

FIG. 3E shows one way to eliminate problems associated with bridge-like structure 202, e.g., by using smaller hole 316. Smaller hole 216 is placed centrally located within obstructive portion 206. This creates a much greater distance 202g between hole 316 and edge 202e than the width of the bridge-like structure 202f shown in FIG. 3E and, therefore, providing bridge 202g with greater structural integrity. The portion of stent 200 corresponding to distance 202g is much less likely to tear, rip, or fracture and cause related problems. FIG. 3F shows another solution to the problem of bridge-like structure 202f, specifically notch 318. Notch 318 is a hole extending from 318a all the way to edge 202e. The C-shape gap in stent 200 shown in FIG. 3F is merely exemplary. Notch 318 may have any suitable shape. As shown in FIG. 3F, notch 318 overlaps with the location of obstructive portion 206 sufficiently to provide flow through stent 200 (i.e., flow F between airway 250 and formerly jailed airway 260 in FIG. 2A). Notch 318's position in FIG. 3F is merely exemplary. For example, notch 318 may fully encompass obstructive portion 206. It may, alternatively, encompass less of obstructive portion 206 than shown in FIG. 3F.

3. Design and Placement of Features Assisting Precise Stent Hole Creation

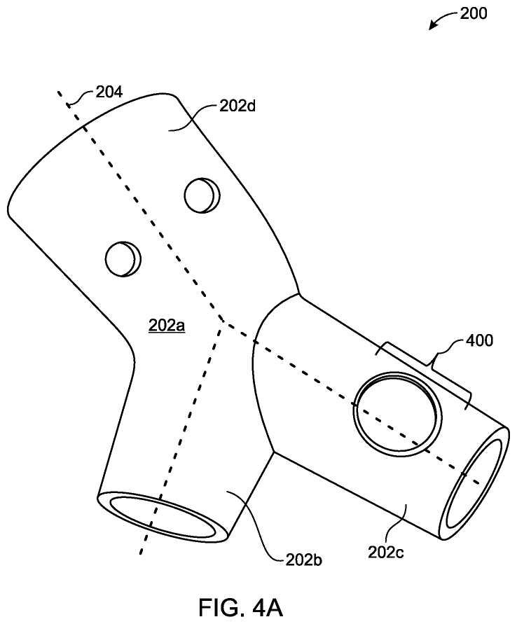

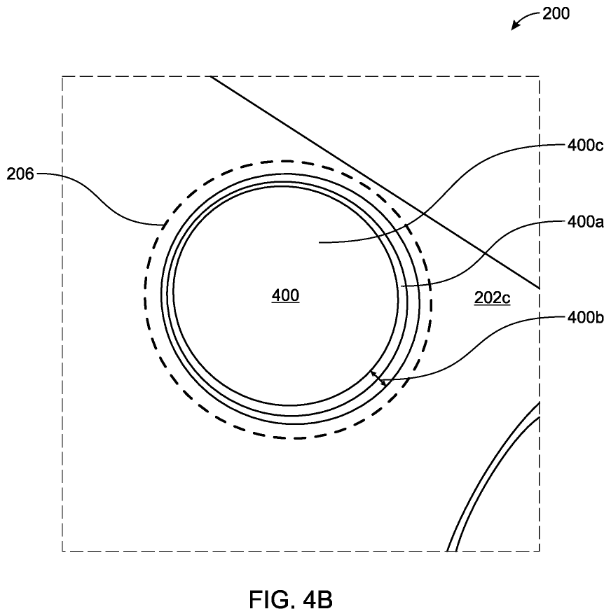

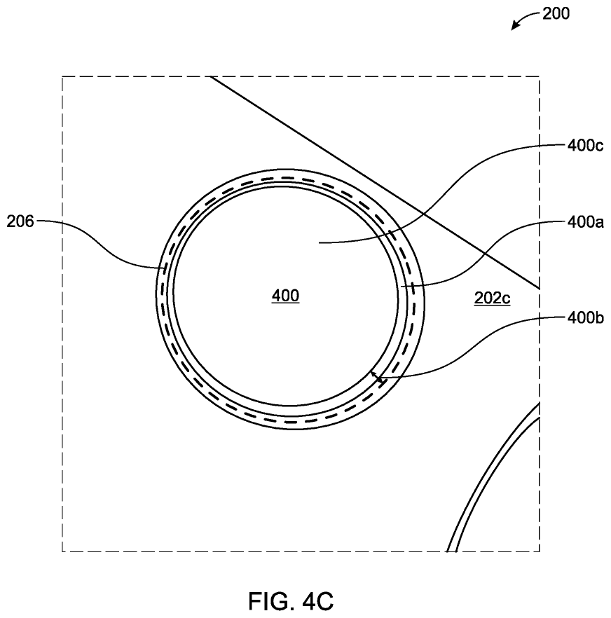

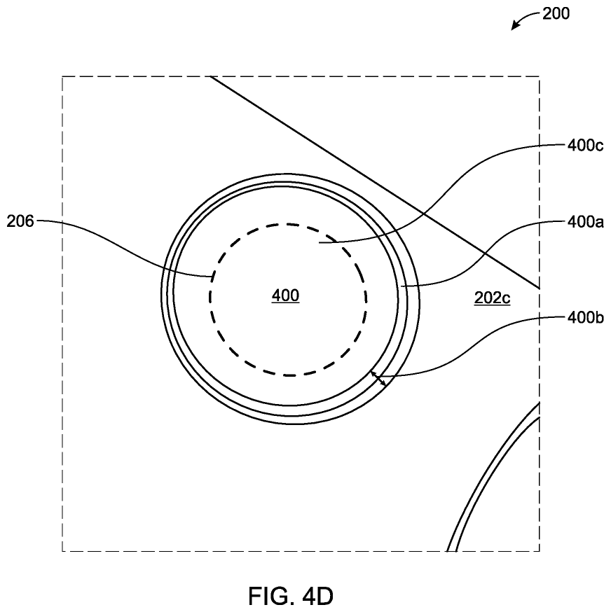

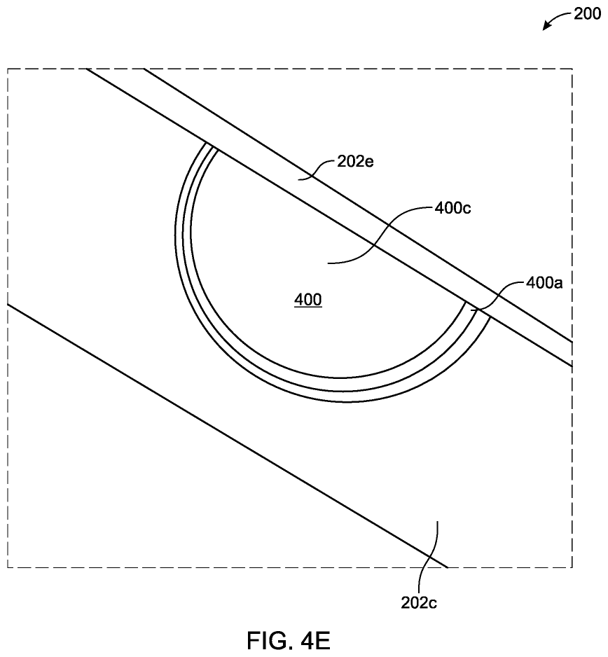

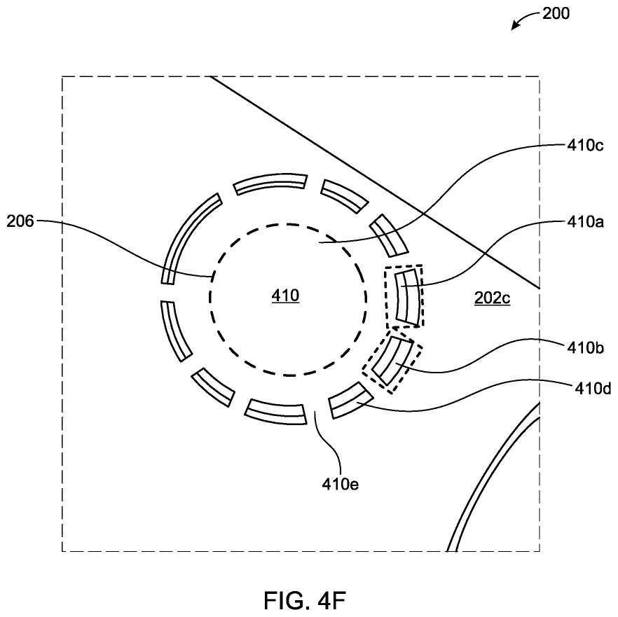

FIG. 4A shows another feature 400 that can assist in opening the stent 200 to flow from jailed airway 260. Feature 400 is shown in more detail in FIG. 4B. As shown in FIG. 4B, feature 400 includes a ring 400a of raised stent material (element 400b shows the height profile of raised material in ring 400a). Ring 400a surrounds a portion 400c of feature 400 that is not raised. Ring 400 may be created by, for example, using the models to add extra thickness to stent 200 in the location of the ring 400a. The extra thickness around ring 400a creates a weakness in the inner circumference of 400a that is directly adjacent to portion 400c. This weakness creates a seam that can be broken once the stent 200 has been 3D printed. Breaking the seam 400a removes portion 400c from the stent 200 thereby opening up portion 400c for airflow. This creates a hole in the stent 200 where portion 400c used to be. Since portion 400c overlaps with obstructive portion 206, breaking the seam (400a) to form the hole allows airflow F between airway 250 and formerly jailed airway 260.

As shown in FIG. 4C, ring 400a can be substantially coincident and circumferential with respect to obstructive portion 206. Ring 400a may coincide exactly with obstructive portion 206. Alternatively, ring 400a may be larger (FIG. 4D) or smaller (FIGS. 4A and 4B) than obstructive portion 206, depending on the particular application. Considerations for sizing of ring 400a and the subsequent hole formed by removing portion 400c are similar, and in some cases identical, to those discussed in the context of sizing holes 310-318 above. Such considerations include, for example, restricting or promoting airflow F and/or prevent the formation of bridge-like portions. Similar and/or the same considerations also apply for the placement of ring 400a with respect to obstructive portion 206 as those discussed above with regard to the relative placement of holes 310-318. The hole formed by removing portion 400c may be placed in different positions with respect to the obstructive portion 206 in order to, for example, prevent the formation of bridge-like portions, etc. Ring 400a may be placed such that the hole formed by removing portion 400c forms a notch towards the edge 202e of stent 200 similar to notch 318 (FIG. 3F). In this case, ring 400 would be placed proximal to 202e such that at least part of portion 400c overlaps with edge 202e. This is shown in FIG. 4E.

FIG. 4F shows another version 410 of a ring construction with perforation. Individual perforations 410a, 410b, and 410d (as well as the others shown in FIG. 4F) may help concentrate stress during punch out of the center portion 410c to make a hole. In particular, differences in thicknesses between the thicker portions 410d of the perforations and the thinner portions 410e of the perforations, causing the stent 200 material to tear more readily upon the application of force. Perforations 410a, 410b, and 410e, etc. can be printed in the same way, for example, as discussed above with respect to ring 400a.

As in the case of ring 410a, perforated ring 410 can be substantially coincident and circumferential with respect to obstructive portion 206. Perforated ring 410 may be designed to coincide exactly with obstructive portion 206. Alternatively, perforated ring 410 may be larger or smaller than obstructive portion 206, depending on the particular application. Considerations for the size of perforated ring 410 and the subsequent hole formed by removing portion 410c are similar to those discussed above concerning the size of holes 310-318 and ring 410a. The hole formed by removing portion 410c can be larger or smaller than obstructive portion 206 in order to, for example, restrict or promote airflow and/or prevent the formation of bridge-like portions. Similar and/or the same considerations also apply for the placement of perforated ring 410 with respect to obstructive portion 206 as those discussed above with regard to holes 310-318 and ring 410a. The hole formed by removing portion 410c may be placed in different positions with respect to the obstructive portion 206 in order to, for example, prevent the formation of bridge-like portions, etc. Similar to the placement of ring 400a in FIG. 3E, perforated ring 410 may be placed such that the hole formed by removing portion 410c forms a notch (not shown but similar to notch 318). In this case, perforated ring 410 would be placed proximally to 202e such that at least part of portion 410c overlaps with edge 202e (not shown).

Stent holes (e.g., holes 310, 312, 314, 316, and 318), notches (e.g., notch 318), and features 400 and 410 are represented above generally with a rounded or circular appearance. While a rounded or circular shape may have certain advantages (e.g., simplicity and symmetry), it is to be understood that these shapes are merely exemplary. Stent holes (e.g., holes 310, 312, 314, 316, and 318), notches (e.g., notch 318), and features 400 and 410 may have any suitable shape. Suitable shapes include slits, triangular or rectangular holes, flaps, x-shapes, etc. One consideration with regard to the shape of features 400 and 410 is the shape of the tool used to punch holes out of them. Features 400 and 410 may, for example, have shapes that mirror the end of this tool, or be shaped to interact with the tool in a specific way. It is also to be understood that any combination stent holes (e.g., holes 310, 312, 314, 316, and 318), notches (e.g., notch 318), and features 400 and 410 may be employed on a stent design. Stent designs may have multiple holds, notches, and features depending on the particulars of the airway in which they are deployed. These features may also be incorporated on portions of the stent with varying thicknesses, as discussed in more detail below. Any of these changes can be accomplished by changing the thickness in the model, and via 3D printing.

### Modifying Other Aspects of the Stent

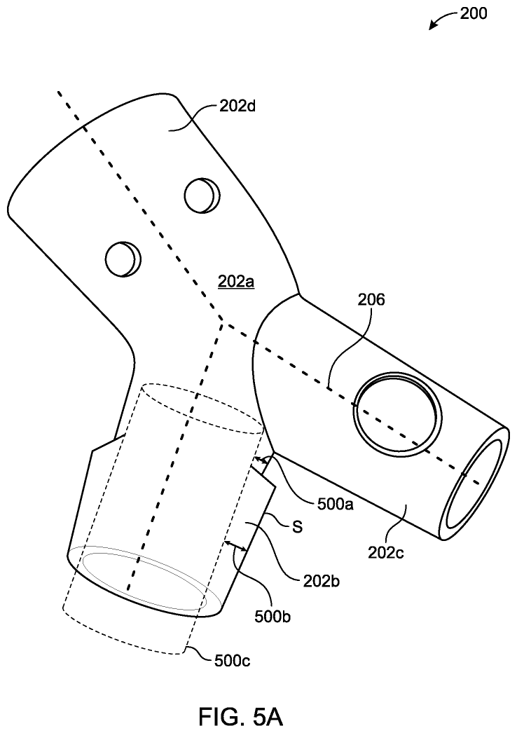

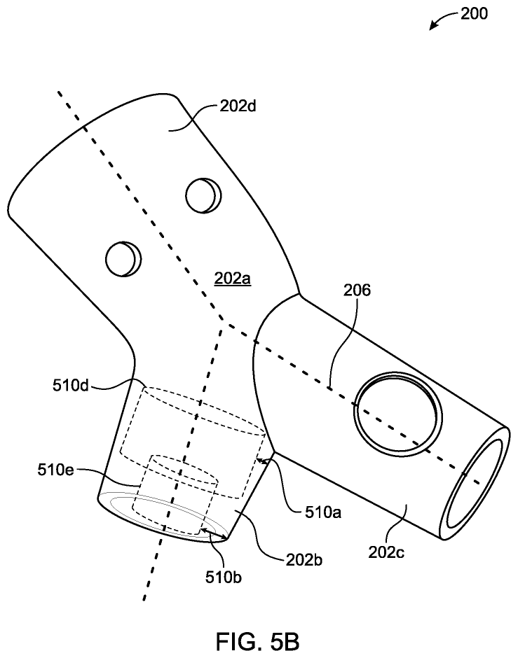

FIG. 5A shows one way a thickness of the printed walls of the stent 200 can vary. As shown in FIG. 5A, the thickness 500a of a portion of stent 200 above position 202b is considerably less than the thickness 500b of a portion below position 202b of the stent 200. Both thicknesses 500a and 500b are shown relative to the outer surface S of the stent and an inner cavity 500c of the stent 200. Thickness 500b may be made greater than thickness 500a in the model design process, as discussed above, prior to printing. In the example discussed above, the user would simply select positions associated with thicknesses 500 and 500b and adjust accordingly.

Different portions of stent 200 may have different thicknesses for different reasons. One reason is that some sections of airway 250 may need more mechanical support than others. For example, portions of airway 250 that have collapsed may need to be supported by an extra strong (thick) portion of stent 200. Portions of the airway 250 showing pathology making them prone to future collapse may also need extra support. Portions of the airway 250 with increased airflow may also need the support of extra thickness. In some cases, it may be advantages to thicken portions of the stent where severe bending or shape change takes place based on the pathology of the patient and the airway locale. Some examples may include progressive malignant (tumor) or benign disease (cyst) where the stent needs to resist the progression of the disease. Lower wall thickness may be required for diseases such as malacia where there is a loss of structure of the airway and the stent is providing more rigidity to the structure rather than resist progressive disease. Although particular portions in FIG. 5A are shown as having an increased thickness, it is to be understood that any other portion of stent 200 may have an increased thickness depending on application. For example, the thickness may be increased or decreased at any of positions 202a-202d. Any of these changes can be accomplished by changing the thickness in the model, and via 3D printing.

The thickness of any region may be a number of percent (e.g., 5, 10, 15%) greater than an average thickness of the overall stent (e.g., thickness 500a shown in FIG. 5A). The thickness of any region may be multiple times the average thickness, e.g., two, three, for, or ten times. In the same way, any portion of stent 200 may have a decreased thickness (not shown). Reasons for decreasing the thickness of a portion include increasing flexibility of the stent at that portion, conserving material (e.g., for weight and cost considerations). The thickness of any region may be less than the average thickness by a number of percent (e.g., 5, 10, 15%). The thickness of any region may be fractions of the average thickness, e.g., one half, one third, one fourth, or one tenth.

FIG. 5A shows increasing the thickness of a portion of stent 200 near location 202b by increasing the thickness in a convex manner (i.e., by thickening the outer wall of stent 200, but leaving the inner cavity 500c the same). This method has the advantage of adding thickness without restricting airflow. However, it may make the stent 200 harder to position during endoscopic placement.

FIG. 5B shows an alternative method of increasing the thickness of stent, again in a lower portion with respect to location 202b. In FIG. 5B, the increase in thickness (compare 510a and 510b) results from decreasing the radius of the interior cavity from 510d in the portion above position 202b to 510e in the portion below position 202b. This decrease in cavity radius results in a greater thickness of stent wall at 510b than 510a, even though the outer wall is not made convex. Advantages to this method of increasing thickness include imparting greater structural stability and strength without rendering stent 200 less maneuverable during deployment. Potential disadvantages may include increased restriction of airflow in the stent.

Although FIGS. 5A and 5B show particular methods of increasing thickness on exemplary portions of stent 200, it is to be understood that these methods can be applied to increase thickness on any other portion of stent 200. Moreover, the two methods are not mutually exclusive and may be applied together. They may also be applied in conjunction with other methods of increasing thickness (e.g., in conjunction with ring 400a formation, etc.) discussed above. They may be applied for the same advantages and reasons discussed above and to the same degree as discussed above.

In addition to the above, increasing thickness of the stent 200 according to any method disclosed herein may be done to help seal off and/or obstruct an airway. For reasons discussed above, it may sometimes be advantageous to obstruct an airway. These reasons include if the airway is somehow compromised and/or functioning in a way that is determinantal to other airways. For example, it may be advantageous to seal off an airway when a lobectomy has occurred and the airway does not terminate into lung but rather pleural space. Sealing an airway may also be advantageous in cases with patients with Chronic obstructive pulmonary disease (COPD) and emphysema localized to specific lung lobes. Regardless of the reasons, increasing the thickness of the stent 200 in the vicinity of the affected airway may be advantageous for obstructing or sealing the affected airway. The thickness may be increased by any method disclosed herein. The deliberately obstructed airways may include jailed airway 260 or any other portion of airway 250 (FIG. 2A).

The openings discussed above (e.g., any of the holes 310, 312, 314, and 316, notch 318, and features used to create holes 400 and 410) may be used to create holes for purposes other than un-jailing jailed airways 260. Other therapeutic modalities, in particular, are contemplated within the scope of the present disclosure. For example, holes created using the above-described techniques may be used for the delivery of medicine, diagnostics, and/or nutrients to portions of the airway.

Multiple stents can be designed for one patient in the same or successive deployments, then interlocked together to form a composite stent architecture. This can be advantageous because designing a single stent for multiple airways is nearly impossible due to difficulty of placement of such a stent.

As shown in FIG. 2A, airways (e.g., airway 250) can be extremely complex. FIG. 2A represents a real airway in a real patient. Airways can vary substantially in angulation, branching, and diameter. The branching may not be dichotomous but rather random and multiple airways may join in one carina all going in different anatomical directions. Airway 250 shows at least eight branches (see, e.g., branches 250a, 250b, 250c, and 260) stemming from the main branch 250. Any of these branches may independently need stents. In many cases, more than one of the branches need stents. Yet, the airway 250 (and airways in patients, more generally) has complex, cavernous interior spaces that do not lend themselves to deploying multi-branch stents. Multi-branch stents require a level of physical complexity that simply cannot, in many cases, be endoscopically deployed.

Instead, multiple stents can be designed with interlocking features according to the principles disclosed above. Stent holes (e.g., holes 310, 312, 314, 316, and 318), notches (e.g., notch 318), and features 400 and 410 can all be designed with complex shapes according to any of the design principles described herein. These complex shapes can include interlocking or fitting portions that allow two or more stents to create a multi-airway structure in situ during endoscopic deployment. These interlocking or fitting components can be designed to form air-tight seals creating a stent superstructure or architecture that dramatically improves flow in the airway in a way separately deployed stents would not. The connecting, interlocking or fitting may be performed inside the patient before, during and subsequent to deployment.

### Preparing and Deploying the Stent

As discussed above, variations 400 and 410 may require punching and removing of portions 400c and 410c, respectively, in order to create a hole in the vicinity of jailed airway 260. Creating this hole prevents the jailing of airway 260. The hole generally needs to be created by applying force to variations 400 and 410, particularly at portions 400c and 410c. As discussed above, the applied force causes stress concentrations in and around 400a and the perforations of 410 that lead to tearing, fracture, and separation of portions 400c to 410c to create a hole.

Punching of 400c and 410c to create holes in the stent to accommodate, e.g., jailed airway 260 may be accomplished by any suitable means. One suitable method is to use forceps (e.g., Dutau Forceps or Lymol Stent Cutting Forceps). However, it should be understood that any suitable hole punching procedure and/or tool can be used. Since, as discussed above, the modeling and printing of the model guides formation of the hole, increased accuracy is achieved. The hole punching can be accomplished prior to deployment or implantation since it is based on accurate models of the airway passages based on diagnostic imaging. In some cases, hole punching may also or alternatively be performed during deployment and/or in situ.

### Overview of Processes and Methods

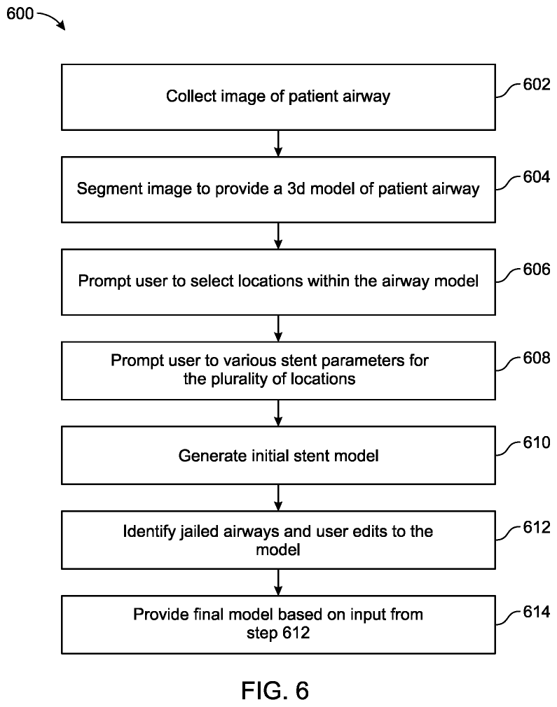

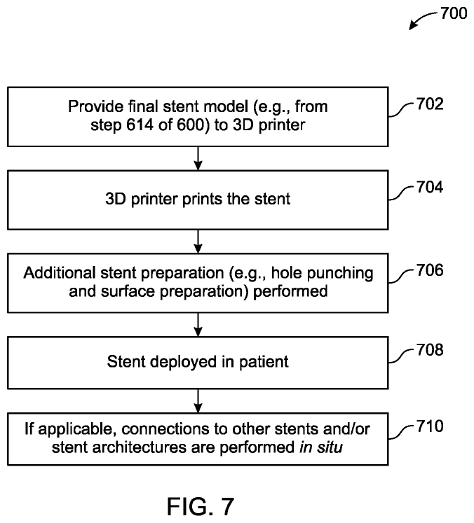

Another aspect of the present disclosure can include methods of generating a stent for a patient's airway, as shown in FIGS. 6 and 7. Methods, processes or algorithms (herein used interchangeably) 600 of FIGS. 6 and 700 of FIG. 7 are illustrated as process flow diagram with flowchart illustrations. For purposes of simplicity, the methods are shown and described as being executed serially. However, it is to be understood and appreciated that the methods in the present disclosure are not limited by the illustrated order as some steps could occur in different orders and/or concurrently with other steps shown and described herein. Moreover, not all illustrated aspects may be required to implement the methods.

Algorithm 600 is a stent design process according to aspects of the present disclosure. In step 602, an image of a patient's airway is collected by any suitable diagnostic technique discussed herein (e.g., CT scan and/or MRI). Step 602 may also analyze and input a generalized airway system for generating a stent model that is not configured for a particular patient. Next, in step 604, the image data collected in step 602 is segmented by CAD software (and/or any other suitable algorithm). A 3D model of the patient (or generalized) airway is then generated based on the segmentation. In step 606, optionally, user input is requested. User review of the airway preempts errors or exaggerations that may arise from imaging noise and/or aberrations. The user also may select, at this point, aspects or locations of the model that would benefit from direct entry of user data. In step 608, the algorithm prompts the user to enter aspects and parameters of the model pertaining to the locations selected in step 606 (e.g., diameters of the mesh model of the stent 202 shown in FIG. 2A). The aspects and parameters may include any of the aspects and parameters disclosed herein, including those pertaining to jailed airway remediation (e.g., any of the methods and techniques disclosed for mediating jailed airway 260). The algorithm then generates an initial stent model in step 610. Either the algorithm and/or the user may identify jailed airways in step 612. In step 612, the algorithm may also prompt the user for other edits to the model (e.g., correction of modeling errors, exaggerations, problems, reinforcements of thickness as discussed in the context of FIGS. 5A and 5B, locations of any holes 310-316, notches 318, and/or features 400 and 410). In step 614, the algorithm incorporates the user edits and any other post-step 610 input information into generating a final model for 3D printing and rapid prototyping (e.g., element 150 of FIG. 1).

FIG. 7 presents a method 700 for printing, preparing, and deploying a stent within aspects of the present disclosure. At step 702 a finalized stent model (e.g., the final model generated in step 614 of method 600) is input to a 3D printing system (e.g., as part of rapid prototyping 150). Any suitable 3D printing system may be used. 3D printing systems that can fabricate shapes with precision using silicone and other inert polymers are preferred. At step 704, the 3D printer prints the stent. The printed stent at this stage may have a number of features (e.g., features 400 and 410) that need additional steps before the stent can be deployed. These additional steps, including punching of holes in features 400 and 410, is performed at step 706. Hole punching features 400 and 410 may be accomplished, as discussed above, by using forceps or any other suitable tool. Hole punching is typically done prior to deployment, i.e., outside of the patient. However, in certain instances, hole punching may be contemplated in situ. Other preparation steps in step 706 may include smoothing any roughened features caused by the 3D printing and stent handling, as well as general surface preparation (e.g., adding coatings for various purposes, including to administer medicine or other therapeutics, to prolong stent life, provide additional reinforcement, and/or to prevent infection). These steps would typically be performed ex situ. In step 708, the stent is deployed inside the patient's airway. Typical deployment uses an endoscopic method. However, any suitable method of stent deployment is within the scope of the present disclosure. Once deployed, in step 710, if applicable, the stent is connected to other stents and/or stent architectures as described above. This step is only applicable when the stent is to be used in conjunction with others. It is performed by interlocking the features of the stents (e.g., holes 310, 312, 314, 316, and 318, notches (e.g., notch 318), and features 400 and 410) such that they join the stents. The joining can be done endoscopically. Typically, joining creates an air-tight seal between the joined stents.

One or more blocks of the flowcharts 600 and 700, and combinations of blocks in the block flowchart illustrations in FIG. 6, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

The methods can be implemented in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the medium for use by or in connection with an instruction execution system. A computer-usable or computer-readable medium may be any non-transitory medium that can contain or store the program for use by or in connection with the instruction or execution of a system, apparatus, or device. As an example, executable code for performing the methods can be stored in a non-transitory memory of a computing device and executed by a processor of the computing device and/or another computing device.

While various inventive aspects, concepts and features of the disclosures may be described and illustrated herein as embodied in combination in the exemplary variations, these various aspects, concepts and features may be used in many alternative variations, either individually or in various combinations and sub-combinations thereof. Unless expressly excluded herein all such combinations and sub-combinations are intended to be within the scope of the present disclosures. Still further, while various alternative variations as to the various aspects, concepts and features of the disclosures—such as alternative materials, structures, configurations, methods, circuits, devices and components, software, hardware, control logic, alternatives as to form, fit and function, and so on—may be described herein, such descriptions are not intended to be a complete or exhaustive list of available alternative variations, whether presently known or later developed. Those skilled in the art may readily adopt one or more of the inventive aspects, concepts or features into additional variations and uses within the scope of the present disclosures even if such variations are not expressly disclosed herein. Additionally, even though some features, concepts or aspects of the disclosures may be described herein as being a preferred arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Still further, exemplary or representative values and ranges may be included to assist in understanding the present disclosure, however, such values and ranges are not to be construed in a limiting sense and are intended to be critical values or ranges only if so expressly stated. Parameters identified as “approximate” or “about” a specified value are intended to include both the specified value and values within 10% of the specified value, unless expressly stated otherwise. Further, it is to be understood that the drawings accompanying the present application may, but need not, be to scale, and therefore may be understood as teaching various ratios and proportions evident in the drawings. Moreover, while various aspects, features and concepts may be expressly identified herein as being inventive or forming part of an disclosure, such identification is not intended to be exclusive, but rather there may be inventive aspects, concepts and features that are fully described herein without being expressly identified as such or as part of a specific disclosure, the disclosures instead being set forth in the appended claims. Descriptions of exemplary methods or processes are not limited to inclusion of all steps as being required in all cases, nor is the order that the steps are presented to be construed as required or necessary unless expressly so stated.

We claim:

1. A bronchial stent comprising:
a first branch configured to at least one of widen, open, and mechanically support a first airway;
an obstructive portion that, when the stent is deployed in the first airway, obstructs a second airway, the second airway forming a branching connection with the first airway; and
a feature proximal to the obstructive portion, the feature configured to facilitate opening of at least a part of the obstructive portion.

2. The stent of claim 1, wherein at least one of:
the feature forms one of a circumference of and an outline of the at least a part of the obstructive portion; and
the stent has an average thickness and the feature has a thickness greater than the average overall stent thickness.

3. The stent of claim 2, wherein the feature thickness is at least one of ten percent more than the average overall stent thickness, twenty percent more than the average overall stent thickness, fifty percent more than the average overall stent thickness, twice the average overall stent thickness, three times the average overall stent thickness, and four times the average overall stent thickness.

4. The stent of claim 1, wherein the feature comprises at least one of a raised portion of the stent, a marked portion of the stent, and a perforation.

5. The stent of claim 4, wherein the perforation substantially outlines the at least a part of the obstructive portion.

6. The stent of claim 1, wherein at least one of:
the facilitating opening of at least a part of the obstructive portion comprises facilitating mechanical removal of the feature from the stent; and
the mechanical removal comprises punching the obstructive portion with an instrument.

7. The stent of claim 6, wherein the instrument is a forceps.

8. The stent of claim 7, wherein the removal is performed prior to deploying the stent in the first airway.

9. The stent of claim 1, wherein at least one of:
the stent is made from a material and the feature comprises the stent material; and
the stent is made from a material and the feature comprises a hole in the stent.

10. The stent of claim 9, wherein the hole at least one of:
substantially overlaps the obstructive portion;
is the at a least part of the obstructive portion; and
substantially encompasses the obstructive portion.

11. The stent of claim 10, wherein the material is silicone.

12. The stent of claim 1, wherein the stent is 3D printed.

13. The stent of claim 12, wherein:
the first and second airway belong to a patient; and
the first branch, the obstructive portion, and the feature proximal to the obstructive portion are configured to substantially fit at least the first airway.

14. The stent of claim 13, wherein the configuring of the first branch, the obstructive portion, and the feature proximal to the obstructive portion comprises designing the first branch, the obstructive portion, and the feature proximal to the obstructive portion using computer aided design.

15. The stent of claim 14, wherein the computer aided design uses at least one of computed tomography (CT) and magnetic resonance imaging (MRI) image of the first and second airways.

16. The stent of claim 15, wherein:
the feature is proximal to an edge of the stent; and
configuring the feature to facilitate opening of at least a part of the obstructive portion comprises designing the feature to avoid creating bridge-like portions in the stent.

17. The stent of claim 16, wherein the designing the feature to avoid creating bridge-like portions comprises creating a notch at the edge of the stent.

18. The stent of claim 1, wherein at least one of:
the first branch, the obstructive portion, and the feature proximal to the obstructive portion are not designed to fit a class of patients; and
the stent further comprises a reinforcing feature.

19. The stent of claim 18, wherein the reinforcing feature has a thickness greater than an average overall stent thickness.

20. The stent of claim 1 wherein at least one of:
the stent further comprises a fitting portion, the fitting portion configured to accommodate another stent;
the accommodating another stent comprises at least one of fitting into a hole in the other stent, connecting to a connecting portion of the other stent, and encompassing an encompassing portion of the other stent;
the accommodating another stent comprises creating an air-tight seal between the stent and the other stent;
the accommodating another stent is accomplished while the stent is inside a patient;
the accommodating another stent creates a stent architecture comprising the stent and the other stent;
the stent architecture comprises more than two stents; and
the stent further comprises an opening configured for a therapeutical purpose;
the therapeutic purpose comprises delivery of medicine.
